(19) United States
(12) Patent Application Publication
(10) Pub. No.: US 2007/0020699 A1
(43) Pub. Date: Jan. 25, 2007

(54) Lateral flow assay and device using magnetic particles

(75) Inventors: Charles R. Carpenter, Scarborough, ME (US); Giosi Farace, Georgetown, ME (US); Paul Scott MacHenry, Portland, ME (US); Brian John Foster, Portland, ME (US); Mirolee Blue Zieba, Westbrook, ME (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: IDEXX Laboratories, Inc., Westbrook, ME (US)

(21) Appl. No.: 11/184,097

(22) Filed: Jul. 19, 2005

(51) Int. Cl. G01N 33/53; G01N 33/551

(52) U.S. Cl. 435/7.5; 436/524; 977/900

(57) ABSTRACT

A complex including magnetic particle bound to a metal colloid. The complex may be part of a reagent for use in a method for determining analytes. The reagent may include a binding partner specific for an analyte. The reagent may further include a first label that is distinguishable from a second label that is used to detect the analyte. The reagent is used in kits and methods for detecting analytes in samples. The methods include immunoassay methods, including method where the first label is used to calibrate the assay.

### BACKGROUND OF THE INVENTION

1. Field of the Invention

In general, the invention relates to the detection of analytes in samples. More specifically, the invention relates to the detection of analytes using devices and kits that include lateral flow matrices and magnetic particles.

2. Description of Related Art

Various analytical procedures and devices are commonly employed in specific binding assays to determine the presence and/or amount of substances of interest or clinical significance which may be present in biological or non-biological fluids. Such substances are commonly termed “analytes” and can include antibodies, antigens, drugs, hormones, etc.

The ability to use materials which specifically bind to an analyte of interest has created a burgeoning diagnostic device market based on the use of binding assays. Binding assays incorporate specific binding members, typified by antibody and antigen immunoreactants, wherein one member of the specific binding pair is labeled with a signal-producing compound (e.g., an antibody labeled with an enzyme, a fluorescent compound, a chemiluminescent compound, a radioactive isotope, a direct visual label, etc.). For example, in a binding assay the test sample suspected of containing analyte can be mixed with a labeled anti-analyte antibody, i.e., conjugate, and incubated for a period of time sufficient for the immunoreaction to occur. The reaction mixture is subsequently analyzed to detect either that label which is associated with an analyte/conjugate complex (bound conjugate) or that label which is not complexed with analyte (free conjugate). As a result, the amount of label in one of these species can be correlated to the amount of analyte in the test sample.

The solid phase assay format is a commonly used binding assay technique. There are a number of assay devices and procedures wherein the presence of an analyte is indicated by the analyte's binding to a conjugate and/or an immobilized complementary binding member. The immobilized binding member is bound, or becomes bound during the assay, to a solid phase such as a dipstick, test strip, flow-through pad, paper, fiber matrix or other suitable solid phase material. The binding reaction between the analyte and the assay reagents results in a distribution of the conjugate between that which is immobilized upon the solid phase and that which remains free. The presence or amount of analyte in a test sample is typically indicated by the extent to which the conjugate becomes immobilized upon the solid phase material.

The use of reagent-impregnated test strips in specific binding assays is also well-known. In such procedures, a test sample is applied to one portion of the test strip and is allowed to migrate or wick through the strip material. Thus, the analyte to be detected or measured passes through or along the material, possibly with the aid of an eluting solvent which can be the test sample itself or a separately added solution. The analyte migrates into a capture or detection zone on the test strip, wherein a complementary binding member to the analyte is immobilized. The extent to which the analyte becomes bound in the detection zone can be determined with the aid of the conjugate which can also be incorporated in the test strip or which can be applied separately.

Other detection technologies employ employing magnetic particles or microbeads, sometimes more specifically termed superparamagnetic iron oxide impregnated polymer beads. These beads bind with the target analytes in the sample being tested and are then typically isolated or separated out of solution magnetically. Once isolation has occurred, other testing may be conducted, including observing particular images, whether directly optically or by means of a camera.

Despite their cost-effectiveness and simplicity of use, typical test strip format assays are less accurate, less precise, and less sensitive to analyte presence than conventional formats. As a result of such drawbacks, the application of test strip format assays has been limited to semi-quantitative or qualitative assays. Among the more significant factors that contribute to the inaccuracy and imprecision of test strip format assays include the manufacture and use of capture lines or spots that bind. It is generally recognized that the manufacture of consistently uniform capture lines requires elaborate material control and manufacturing processes with rigid specifications that must operate within narrow tolerances. Moreover, to function properly, most test strip formats require that the analytes to be detected must be uniformly captured in a precise geometry at a precise location on the test strip and that factors such as the ambient humidity present at the time of test strip manufacture, type of membrane utilized in such manufacturing process, and a capture reagent-receptor itself contributing greatly to assay inaccuracies and false readings.

Accordingly, what is needed is a method and device for detecting analytes that has the cost-effectiveness and simplicity of use of a test strip, but with the accuracy and precession required for quantitatively detecting small amounts of analytes.

### SUMMARY OF THE INVENTION

In one aspect, the invention is directed to a complex of a magnetic nanoparticle bound to a metal colloid. The magnetic nanoparticle may include an iron oxide and a polymer and have a diameter of about 50-1000 nanometers. The metal may be gold, silver or a rare earth metal. A first label, for example a fluorescent metal chelate, may be attached to the colloid. Either the nanoparticle or the colloid may include an analyte-specific binding partner. The complex, labels and binding partners can be combined in various ways to provide a particulate reagent for detecting analytes.

In another aspect, the invention is directed to a device and a kit for detecting an analyte. The device includes a porous carrier matrix that has a detection zone including a magnet and an average pore size that allows for the substantially unimpeded lateral flow of the particulate reagent, which includes the complex of the magnetic particle bound to the metal colloid. The magnet may be associated with the detection zone so that a magnetic field attracts and substantially confines the reagent in the detection zone when the reagent is applied to the matrix. The matrix may also include a sample application zone laterally spaced from the detection zone. The kit includes a conjugate reagent having a second label bound to an analyte-specific binding partner or to an analog of the analyte. The signal from the second label is distinguishable from a signal from the label that is attached to the colloid.

In yet another aspect, the invention is directed to a method for determining the presence or amount of an analyte in a sample. Methods include both sandwich and competition immunoassay formats. For example, a competition method includes forming a mixture of the sample, a particulate reagent having a first label, and a conjugate reagent of a label and a specific binding partner for the analyte or an analog of the analyte. The mixture is contacted with a device having a porous carrier matrix having detection zone including a magnet. The matrix has an average pore size that allows for the substantially unimpeded lateral flow of the reagent. The method further includes washing the mixture from the matrix in the area of the detection zone and simultaneously or sequentially detecting a signal from the first label and the second label in the detection zone to determine the presence or amount of the analyte in the sample.

Still another aspect of the invention includes a method for calibrating an assay for detecting an analyte in a sample, wherein the assay includes contacting the sample with a conjugate reagent having a label attached to an analyte-specific binding partner or an analog of the analyte. The method includes forming a mixture of the sample with a particulate reagent having a label. The mixture is contacted with a device having porous carrier matrix having a detection zone that includes a magnet. The porous carrier has an average pore size that allows for the substantially unimpeded lateral flow of the particulate reagent. The amount of the signal associated with label of the particulate reagent in the detection zone is measured, thereby calibrating the assay.

### BRIEF DESCRIPTION OF THE FIGURES

FIG. 1 is a graph showing the correlation of the chemiluminescence and fluorescence signals associated with the method of the invention.

FIG. 2 is a graph showing the use of fluorescence to monitor effect of rehydration volume on chemiluminescence activity of lyophilized particles

FIGS. 3A and 3B are graphs showing the results of the titration of Urea H₂O₂ with a HRP-TRF particle of the present invention. FIG. 3A shows that there was a linear relationship between the chemiluminescence and concentration of the urea peroxide, and there was no cross talk between the two signals. In FIG. 3B, the consistent fluorescence shows consistent dispensing and capture efficiency in the assay.

FIG. 4 shows the correlation of the FeLV dose response to the concentration of H₂O₂.

FIG. 5 is a graph showing the sensitivity of an FeLV assay using the invention.

FIG. 6 is a series of graphs showing the results of 60 assays for FeLV using the invention.

FIG. 7 is a graph showing the results of a T4 assay in a competitive format at various concentrations of T4 using the invention.

### DETAILED DESCRIPTION

The invention provides devices, kits and methods for conducting specific binding-pair assays using the principle of lateral flow through a porous carrier matrix for the qualitative or quantitative analysis of selected analytes in samples. The invention can be used for a wide variety of assays, both ligand-based and non-ligand-based. Applicable ligand-based methods include, but are not limited to, competitive immunoassays, non-competitive or so-called sandwich technique immunoassays, and blocking assays. The use of the invention is not limited to any particular analyte. The embodiments described herein are solely for illustrative purposes and are not intended to limit the scope of the invention to any particular set of binding partners or assay format.

The invention involves magnetic particles having bound thereto analyte-specific binding reagents and/or labels. In addition, the invention provides for a porous carrier matrix that allows for the unimpeded flow of magnetic particles suspended in a carrier liquid, such as the sample or other liquid reagent. The porous carrier matrix allows for the lateral flow of the liquid containing the magnetic particles to a point in the matrix associated with a magnet that stops the flow of the particles but not the liquid. As the liquid carrying the magnetic particles passes through the magnetic field, the particles are attracted to the field and form a detection zone in discreet location on the matrix. The analyte is captured in the detection zone and detected with a label.

In one aspect, the particles include a label that is independent and different than a label used to detect the analyte. The label associated with the particle allows for internal calibration of the assay by allowing for the determination of the amount of analyte binding reagent present in the detection zone on the porous carrier. The label associated with the particle and a label used to detect the analyte can be simultaneously or sequentially detected. The amount of a signal corresponding to the label associated with the particle can be used to quantitatively determine the amount of the analyte associated with a signal produced by the label used to detect the analyte.

Because analyte specific reagents do not need to be covalently attached and dried on the porous carrier matrices, the reactivity and variance of the reagents can be controlled to a greater extent than systems that require such techniques. In addition, the label associated with the particle allows for the accurate detection of analyte quantity by compensating for multiple variables including those involving the efficiency of the particle flow through the matrix, the efficiency of the particle capture at the magnet, and the variability in dilutions and pipetting volumes. Similarly, variables that may result from lyophilization of reagents, such as the variability in the amount of analyte binding reagent or other reagent used in the detection of the analyte (e.g. an analyte analog), can be accounted for by comparing the amount of the signal from the particle to the amount of signal associated with the analyte detection.

Before describing the present invention in detail, a number of terms will be defined. As used herein, the singular forms “a,” “an”, and “the” include plural referents unless the context clearly dictates otherwise.

By “analyte” is meant a molecule or substance to be detected. For example, an analyte, as used herein, may be a ligand, which is mono- or polyepitopic, antigenic or haptenic; it may be a single compound or plurality of compounds that share at least one common epitopic site; it may also be a receptor or an antibody.

A “sample” refers to an aliquot of any matter containing, or suspected of containing, an analyte of interest. For example, samples include biological samples, such as samples from taken from animals (e.g., saliva, whole blood, serum, and plasma, urine, tears and the like), cell cultures, plants, etc.; environmental samples (e.g., water); and industrial samples. Samples may be required to be prepared prior to use in the methods of the invention. For example, samples may require diluting, filtering, centrifuging or stabilizing prior to use with the invention. For the purposes herein, “sample” refers to the either the raw sample or a sample that has been prepared.

“Binding specificity” or “specific binding” refers to the substantial recognition of a first molecule for a second molecule, for example a polypeptide and a polyclonal or monoclonal antibody, an antibody fragment (e.g. a Fv, single chain Fv, Fab′, or F(ab′)2 fragment) specific for the polypeptide, enzyme—substrate interactions, and polynucleotide hybridization interactions.

“Non-specific binding” refers to non-covalent binding between molecules that is relatively independent of specific surface structures. Non-specific binding may result from several factors including electrostatic and hydrophobic interactions between molecules.

“Member of a specific binding pair” or “specific binding partner” refers one of two different molecules, having an area on the surface or in a cavity which specifically binds to and is thereby defined as complementary with a particular spatial and polar organization of the other molecule. The members of the specific binding pair are referred to as ligand and receptor (antiligand). These will usually be members of an immunological pair such as antigen-antibody, although other specific binding pairs such as biotin-avidin, hormones-hormone receptors, IgG-protein A, polynucleotide pairs such as DNA-DNA, DNA-RNA, and the like are not immunological pairs but are included in the invention and the definition of specific binding pair member.

“Analyte-specific binding partner” refers to a specific binding partner that is specific for the analyte.

“Substantial binding” or “substantially bind” refer to an amount of specific binding or recognizing between molecules in an assay mixture under particular assay conditions. In its broadest aspect, substantial binding relates to the difference between a first molecule's incapability of binding or recognizing a second molecule, and the first molecules capability of binding or recognizing a third molecule, such that the difference is sufficient to allow a meaningful assay to be conducted distinguishing specific binding under a particular set of assay conditions, which includes the relative concentrations of the molecules, and the time and temperature of an incubation. In another aspect, one molecule is substantially incapable of binding or recognizing another molecule in a cross-reactivity sense where the first molecule exhibits a reactivity for a second molecule that is less than 25%, preferably less than 10%, more preferably less than 5% of the reactivity exhibited toward a third molecule under a particular set of assay conditions, which includes the relative concentration and incubation of the molecules. Specific binding can be tested using a number of widely known methods, e.g., an immunohistochemical assay, an enzyme-linked immunosorbent assay (ELISA), a radioimmunoassay (RIA), or a western blot assay.

“Ligand” refers any organic compound for which a receptor naturally exists or can be prepared.

“Analyte analog” or an “analog of the analyte” refers to a modified form of the analyte which can compete with the analyte for a receptor, the modification providing means to join the analyte to another molecule. The analyte analog will usually differ from the analyte by more than replacement of a hydrogen with a bond that links the analyte analog to a hub or label, but need not. The analyte analog can bind to the receptor in a manner similar to the analyte.

“Receptor” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, protein A, complement component C1q, and the like.

“Antibody” refers to an immunoglobulin that specifically binds to and is thereby defined as complementary with a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

Porous Carrier Matrix

The invention employs a porous carrier matrix capable of providing lateral flow to a liquid test sample and/or liquid reagents. Generally, the porous carrier matrix can be selected from any available material having appropriate thickness, pore size, lateral flow rate, and color. Lateral flow refers to liquid flow in which all of the dissolved or dispersed components of the liquid are carried at substantially equal rates and with relatively unimpaired flow laterally through the matrix, as opposed to the preferential retain of one or more components of the liquid, such as a chromatographic separation of the sample. Examples of suitable porous carrier matrices include glass fiber mats, non-woven synthetic mats, sintered particulate structures, cast or extruded matrix materials, or other materials characterized by the presence of adhesion within the material. These materials may be a formed (molded or cast) from open pore structures such as nylon or nitrocellulose. The porous carrier matrix may also be a particulate material such as glass particles or polymer particles.

The porous carrier matrix may be made from a material which has a low affinity for the analyte and test reagents. This is to minimize or avoid pretreatment of the test matrix to prevent nonspecific binding of analyte and/or reagents. However, materials that require pretreatment may provide advantages over materials that do no require pretreatment. Therefore, materials need not be avoided simply because they require pretreatment. Hydrophilic matrices generally decrease the amount of non-specific binding to the matrix.

In one aspect, the porous carrier matrix has an open pore structure with an average pore diameter of 1 to 250 micrometers and, in further aspects, about 3 to 100 micrometers, or about 10 to about 50 micrometers. The matrixes are from a few mils (0.001 in) to several mils in thickness, typically in the range of from 5 or 10 mils and up to 200 mils. The matrix should be translucent to allow for the visualization or photometric determination of the light and or color throughout the thickness of the matrix. The matrix may be backed with a generally water impervious layer, or may be totally free standing.

An example of a suitable porous carrier matrix in which lateral flow occurs is the high density or ultra high molecular weight polyethylene sheet material manufactured by Porex Technologies Corp. of Fairburn, Ga., USA. This material is made from fusing spherical particles of ultra-high molecular weight polyethylene (UHWM-PE) by sintering. This creates a porous structure with an average pore size of eight microns. The polyethylene surface is treated with an oxygen plasma and then coated with alternating layers of polyethylene imine (PEI) and poly acrylic acid (PAA) to create surfactant-free hydrophilic surface having wicking rate of 70 sec/4 cm.

While matrices made of polyethylene have been found to be highly satisfactory, lateral flow materials formed of other olefin or other thermoplastic materials, e.g., polyvinyl chloride, polyvinyl acetate, copolymers of vinyl acetate and vinyl chloride, polyamide, polycarbonate, polystyrene, etc., can be used. Examples of suitable materials include Magna Nylon Supported Membrane from GE Osmonics (Minnetonka, Minn.), Novylon Nylon Membrane from CUNO Inc. (Meriden, Conn.) and Durapore Membrane from Millipore (Billerica, Mass.).

The matrix materials may be slit, cut, die-cut or punched into a variety of shapes prior to incorporation into a device. Examples of alternative shapes of the matrix include circular, square/rectangular-shaped, flattened ellipse shaped or triangularly shaped. While not a focus of the invention, if desired, biological reagents may be applied to the materials before or after forming the desired shape. Biological reagents may be attached to the materials by any available method, for example, either by passively, diffusively, non-diffusively, by absorption, or covalently, depending upon the application and the assay.

Magnetic Nanoparticles

The invention employs a magnetic nanoparticle that is separable from solution with a conventional magnet. Such particles are usually provided as magnetic fluids or ferrofluids, as they are often called, and mainly consist of nano sized iron oxide particles (Fe₃O₄ or γ-Fe₂O₃) suspended in carrier liquid. Although generally characterized as magnetic, the particles of the invention include superparamagnetic particles, meaning that these particles can be easily magnetized with an external magnetic field and redispersed immediately once the magnet is removed. The particles should have a small size distribution and uniform surface properties.

The magnetic particles can be prepared by a variety of established methods, generally by the precipitation of iron oxide in the presence of polymers, coating of iron oxide with polymers according to the core-shell principle, or high pressure homogenization. The precipitation methods generally provide particles having a diameter of 50-100 nanometers, the core shell method provide particles with diameters from about 200 to 500 nanometers, and the homogenization technique generally provides particles between those ranges.

For example, particles may be prepared by superparamagnetic iron oxide by precipitation of ferric and ferrous salts in the presence of sodium hydroxide and subsequent washing with water. The size of the iron oxide cores can be determined by photon correlation spectroscopy (PCS). The iron oxide cores can be coated with polysaccharides such as dextran, starch, chitosan, and ficell, or with synthetic polymer polyethylene imine and polyvinylpyrrolidine at temperatures depending on the solubility of the polymers in water. Generally, the molecular weight of the polymer corresponds to the size of the magnetic particle. The temperature and strength of the applied base for the iron oxide precipitation can influence the size of the particles and their percentage of iron oxide within the particle. The polymer used can also influence the amount of iron oxide in the particle. See Grüttner, C. et al., Preparation and Characterization of Magnetic Nanospheres for In Vivo Application, in Scientific and Clinical Applications of Magnetic Carriers, Haefeli, et al., Eds., Plenum Press, New York, 1997, pp. 53-67.

In another example, biodegradable magnetic nanoparticles which includes a mixture of non-toxic biodegradable magnetic metal oxide nanophases (i.e. Fe₃O₄ or γ-Fe₂O₃) and active t-PA, is packed in biodegradable poly(lactic acid) (PLA) nanospheres having sizes ranging from 100 nm to several microns. PLA nanospheres are encapsulated with poly(ethylene glycol) (PEG) to provide protection against non-specific binding with sample components.

The magnetic particles can be functionalized to provide a surface for coupling the particles to a molecule or biomolecule. Depending upon the polymer, the surfaces can be activated with a variety of functional groups readily known to those skilled in the art. These groups include, for example, amino, carboxy, alcohol, and aldehyde groups. In one aspect of the present invention, the particle is attached to a metal colloid. A variety of attachment chemistries can be used, including covalent attachment or attachment through specific binding partners. Linking molecules may also be employed.

Currently available formats of particles can be broadly classified into unmodified or naked particles, chemically derivatized particles with general specificity ligands (streptavidin, Protein A, etc) and chemically derivatized particles with specific recognition groups such as monoclonal and polyclonal antibodies. Suitable particles with diameters ranging from 50 to 1000 nanometers, and functionalized with a variety surfaces, are available from a number of sources including Micromod Partikeltechnologie GmbH, Rostock-Warnemuende, Germany, Ademtech, Parc scientifique Unitec 1, 4. Allee du Doyen George Brus, 33600 Pessac, France, and EMD Biosciences Inc., Estapor® Microspheres, Division Life Science Products, 1658 Apache Dr. Naperville, Ill. (USA).

Labels

In one aspect the assay method of the invention employs two labels. The first label is associated with the magnetic particle used in the invention. The second label is part of a conjugate reagent that includes the label and an analyte-specific binding partner or an analyte analog. A “label” is any molecule that is bound (via covalent or non-covalent means, alone or encapsulated) to another molecule or solid support and that is chosen for specific characteristics that allow detection of the labeled molecule. Generally, labels include, but are not limited to, the following types: particulate metal and metal-derivatives, radioisotopes, catalytic or enzyme-based reactants, chromogenic substrates and chromophores, fluorescent and chemiluminescent molecules, and phosphors. The utilization of a label produces a signal that may be detected by means such as detection of electromagnetic radiation or direct visualization, and that can optionally be measured.

In one aspect on the invention, the magnetic nanoparticle is attached to a metal colloid. In typical immunoassays, colloidal gold provides a label for visual or qualitative detection. In the primary aspect of the present invention, however, the colloid serves as a carrier for a different label. To avoid confusion, the label carried by the metal colloid will be referred to as a label attached to colloid to distinguish this label from the colloid itself. Recently, for example, colloidal gold particles have been coated with fluorescent metal chelates. U.S. Patent Application Publication No. 20040082768, which is incorporated by reference herein its entirely, describes Europium chelates coated onto colloidal gold particles. Also, the colloid may be coupled to other conventional and unconventional fluorescent, chemiluminescent and enzyme labels, including for example, fluoresceins, rhodamines, Texas Red, luminol, anthracenes, luciferaces, peroxidases, dehydogenases, and others. While the possibilities for the label attached to the colloid are extensive, the label must produce a signal that is distinguishable from signal produce by the label of the conjugate reagent. In one aspect of the invention, the label should be capable of producing a signal in an aqueous environment that is a quantifiable, preferably by conventional instrumentation.

Metal colloids are primarily gold and silver, but other metals, especially rare earth metals, are appropriate. The size of the colloid is not significant as long as the colloid and the magnetic particle, when attached, are capable of remaining in suspension in carrier liquids.

In certain aspects of the invention, the label attached to the colloid includes a chelate of a tripositive lanthanide ion, such as Eu³⁺, Tb³⁺ and Sm³⁺. These chelates may include chelating ligands, enhancing ions, and synergistic agents to increase the intensity of the signal from the label. In certain aspects, the label attached to the colloid operates on the principle of time-resolved fluorimetry to eliminate the effects of background signal and increase the sensitivity of the assay.

The fluorescent properties of tripositive lanthanide ions, especially the chelates of Eu³⁺, Yb³⁺ and Sm³⁺, are particularly well suited for time-resolved fluorimetry. In these chelates, a strong ion emission originates from an intrachelate energy transfer, where an organic ligand absorbs the excitation radiation in the ultraviolet (UV) range and transfers the excited energy to the emitting ion. The ligand field around the ion also prevents the quenching caused by coordinated water molecules which in aqueous solution tend to create an efficient deactivation route. The ion-specific emission appears at narrow banded lines at long wavelengths (Tb³⁺ 544 nm, Eu³⁺ 613 nm, Sm³⁺ 643 nm) with a long Stokes' shift (230-300 nm). The most important feature in this context is the long fluorescence lifetime, ranging from 1 μs to over 2 ms, which makes it possible to apply time-resolved detection for the effective elimination of the background and to increase the sensitivity.

β-Diketones have gained the widest use as the ligands to increase lanthanide fluorescence. As bidentate chelating agents, they form relatively stable chelates; the six-membered ring involved in the chelate structure directly absorbs the excitation light and efficiently transfers the energy to the chelated ion. For some chelates, however, fluorescence is essentially limited to organic solvents, making them unattractive or impractical for biological applications. In one aspect of the invention, the label attached to the colloid is a β-diketone that forms a fluorescent chelate with lanthanide (III) rare earth metal ions in an aqueous solvent as described in U.S. patent application No. 20040082768. This label can be detected by irradiating the label with energy equal to or greater than 360 nm and detecting the fluorescence from label. In addition, several other β-diketones are known effective chelating agents as described in Xu, Y, et al., Co-fluorescence Effect in Time-resolved Fluoroimmunoassay. A Review, Analyst 1992 (117:1061-1069):
Thenoyltrifluoroacetone (TTA)
Benzoyltrifluoroacetone (BTA)
2-Furoyltrifluoroacetone (FTA)
p-Fluorobenzoyltrifluoroacetone (FBTA)
β-Naphthoyltrifluoroacetone (β-NTA)
1,1,1,2,2-Pentafluoro-5-phenylpentane-3,5-dione (PFPP)
Dibenzoylmethane (DBM)
Di-p-fluorobenzoylmethane (DFBM)
Pivaloyltrifluoroacetone (PTA)
1,1,1-Trifluoro-6-methylheptane-2,4-dione (TFMH)
Dipivaloylmethane (DPM)
1,1,1,5,5,5-Hexafluoroacetylacetone (HFAcA)
1,1,1,2,2-Pentafluorohexane-3,5-dione (PFH)
1,1,1,2.2-Pentafluoro-6,6-dimethylheptane-3,5-dione (PFDMH)
1,1,12,2,2,3,3-Heptafluoro-7,7-dimethyloctane-4,6-dione (HFDMO)
1,1,1,2,2-Pentafluorotetradecane-2,4-dione (PFTD)
1,1,1-Trifluorotridecane-2,4-dione (TFTD)
1,1,1-Trifluoroacetylacetone (TFAcA)
Acetylacetone (AcA)

In many instances, the strong fluorescence intensity of lanthanide β-diketone chelates requires a non-aqueous chelate environment. For instance, the three β-diketone molecules in a tris-chelate occupy only six of the nine available coordination sites of the ion, which still remains sensitive to quenching by water. Synergist ligands have been introduced to work as an ‘insulating sheet,’ in which a synergistic ligand (or a synergistic agent) is involved in the chelate structure to replace water molecules from the ligand field. The agent can act as a shield, protecting the chelate from external interactions and thus efficiently reducing non-radioactive energy degradation. Examples of synergistic ligands applicable for co-fluorescence enhancement are 1,10-phenanthroline (Phen) and its derivatives, e g, 4,7-(or 5,6)-dimethyl-1,10-phenanthroline, 4,7-diphenyl-1,10-phenanthroline, 2.9-dimethyl-4,7-diphenyl-1,10-phenanthroline, pyridine derivatives such as 2,2′-dipyridyl (DP), 2,2′-dipyridylamine, 2,4,6-trimethylpyridine, 2,2′:6′, 2″-tetrapyridine and 1.3-diphenylguanidine. Tri-n-octyl-phosphene oxide (TOPO) has been found to be one of the most effective synergistic agents. Usually, the fluorescence on the lanthanide chelate increases with increasing concentration of the synergistic ligand until a maximum and almost stable fluorescence is reached.

In another aspect, the fluorescent label may be used in the presence of enhancing ions. The enhancing ions generally applied are Gd³⁺, Tb³⁺, Lu³⁺, La³⁺ and Yb³⁺. Enhancing ion are useful because, under some conditions involving time resolved fluorimetry, a weak co-fluorescence enhancement effect is obtained, for example with Yb³⁺ and Dy³⁺. This is because, in the co-fluorescence enhancement system, the enhancing ion used must not have excited 4f or 4d levels situated below the excited triplet level of the β-diketone used. Hence the energy absorbed by these chelates cannot be dissipated through these non-existing energy levels. Instead, the energy is transferred to the fluorescent ions through an intermolecular energy transfer. Enhancing ions are needed to provide the high molar excess of the triplet sensitizer to ensure a linear response for the acceptor detection. The high concentration is also needed to create the aggregates for necessary for energy transfer.

Non-ionic detergents have been found to protect the chelates against non-radioactive processes and solubilize the chelates and stabilize the solutions by preventing sedimentation. Examples of suitable detergents include TRITON® X-100 and TWEEN® 20. Water soluble organic solvents may also be used to enhance fluorescence.

In another aspect of the invention, a conjugate reagent includes a label for detecting the analyte. This label is attached to an analyte-specific binding partner or an analyte analog. This label is independent and distinct from the label attached to the colloid and its signal must be distinguishable from the label attached to the colloid. In various aspects of the invention, the two labels are detected sequentially or simultaneously. The attachment of the labels to the analyte-specific binding partners may be accomplished directly, through a linker, or through a pair of specific binding partners (e.g. biotin/avidin) as is well known in the art. If the label on the conjugate reagent and the label attached to the colloid are both fluorescent labels, then the labels should distinguishable, for example by excitation at different wavelengths or by different emission spectra.

In one aspect, the invention is directed to a reagent for detecting an analyte in a sample that includes a metal colloid with a label attached to the colloid. The reagent may also include one or more of an analyte-specific binding partner, a protein or another (non-analyte) specific binding partner such as an antibody, antigen, streptavidin or biotin. The (non-analyte) specific binding partner and the protein may be used to couple the colloid to the magnetic particle, either via a complementary binding partner on the particle or through an appropriate linker molecule.

Particulate Reagent

One aspect of the invention is directed to a particulate reagent that includes a complex of the magnetic particle and the metal colloid. In this aspect, the metal colloid is functionalized with a label attached to the colloid.

The colloid and the particle may be attached using well known linking chemistries. These chemistries include, for example, direct attachment using zero linkers such as EDC, and long chain linkers, or indirectly through a pair of binding partners such as biotin and avidin/streptavidin/neutravidin. To assist in the linking, the magnetic particle may be functionalized with amino, aldehyde, hydroxyl, or carboxyl groups.

Colloidal gold has been used extensively as a label in immunoassays. Therefore methods for functionalizing the colloids and attaching them to various molecules are well characterized. For example, U.S. Patent Application Publication No. 20040082768 describes a gold colloid coated with a Europium chelate and a bound to an antibody.

In another aspect of the invention, the colloid includes an analyte-specific binding partner. When label and binding partners are added to the colloid together they are added sequentially, label first. In general, the specific concentrations of the label and binding partners are kept the same but concentrations may vary depending on the binding partners. When all three components (i.e., the label, the specific binding partner and the analyte-specific binding partner) are included on the colloid, the components are added in the following order: label, analyte-specific binding partner, and specific binding partner.

In general, the particulate reagent includes the label attached to the colloid. A second label associated with the conjugate reagent is used to detect the presence or amount of analyte in the sample. In one embodiment, however, the second label is attached (either through a linking group or through a pair of binding partners) to the colloid or the particle. Thus, this reagent will provide two signals regardless of the presence of the analyte in the sample. Accordingly, this reagent can be used as a control to determine the quality of other reagents in the assay, such as a substrate reagent.

In one aspect, the particulate reagent is provided in a lyophilized form that allows for their convenient use in a commercial assay. The particulate reagents may be lyophilized by standard lyphilization protocols. In general, the particulate reagent is mixed with trehalose (or any other cyroprotectant) and dispensed into individual vials. They are then lyophilized using a standard lyophilization protocol and stoppered under either vacuum or dry nitrogen. To reduce meltback post lyphophilization, the lyophilized material is dried at elevated temperatures for an extended period of time following the completion of the lyophilization cycle; for example, at at 45° C. for at least 60 hours.

Devices

In another aspect, the invention provides for a device for detecting the presence of an analyte in a sample. The device includes a porous carrier matrix and a magnet. In an example of the operation of the device, a solution containing the sample, the particulate reagent and a detection reagent labeled with an appropriate reporter molecule is applied to the device. The magnet is associated with the porous carrier matrix so the magnetic field will attract and substantially retain the particles at a discreet location on the matrix.

The shape of porous carrier is important only to the extent that it provides a suitable format for conducting the assay. In its simplest form, the carrier is a strip having a sample application zone and a detection zone, which may be the same zone. The magnet is associated with the detection zone so that the magnetic field defines the zone such that the field attracts and detains the magnetic particles in the detection zone. For example, the magnet can be located underneath the porous carrier so that the lateral flow of the particles in a solution flowing past the detection zone is stopped. While the solution continues to traverse the matrix, the particles will be held in place by the magnetic field.

The magnetic may be held directly against the matrix or spaced apart from the matrix to any extent so long as the magnetic field in the detection zone is strong enough to prevent substantially all of the particles from passing through the detection zone. “Substantially all” means that the magnet prevents the particles from passing through the detection zone so that the sensitivity of the assay is not substantially different than if all of the particles had been detained in the detection zone. Substantially all the particles is at least a majority of the particles, and preferably at least about 70%, about 80%, about 90%, or about 95% of the particles should be retained in the detection zone, and more preferably about 99% or more are retained.

The magnetic may be held in place by an adhesive, a clamp or any other device that keeps the magnet in place in the detection zone. In one aspect, the porous carrier matrix is held within a rigid body for convenient handing by the operator. The magnet and the matrix may be held in place within the rigid body so that the location of the magnet is fixed in relation to the matrix. The material for the rigid body is unimportant as long as it is compatible with the sample and reagents, and does not affect the magnetic field. Preferably, the body will be readily and inexpensively manufactured and packaged.

The device may include a sample application zone, which may be laterally spaced from the detection zone. The sample application zone may include a separate pad, cup, well or other member that facilitates the application of the sample solution and/or other reagents at a discreet location on the matrix. The sample application zone may also include a conjugate reagent non-diffusively bound the matrix, a separate pad or other member so that the reagent is solubilized by the sample solution upon addition of the solution.

Generally the shape of the matrix should be suitable for the analyte or analytes being detected. The matrix may include two or more discreet channels that for measuring multiple analytes in a sample. The matrix may be divided physically, such as by providing fluid impermeable barriers between the channels, or by treating discreet areas of the matrix with reagents that render the matrix impermeable to liquid flow. For example, a fluoro methacrylate polymer can be used to create discreet channels in the matrix by rendering portions of the matrix impermeable to liquid.

The shape of the detection zone is generally defined by the shape of the magnetic field, which is generally the shape of the magnet. In one example, the matrix is a strip with and the magnet is a circular disk located under the strip. In operation, the detection zone will take a circular shape of the surface of the matrix. Other shapes should be compatible with the shape of the matrix and the number of analytes under consideration. It has been found that when the magnetic particles are deposited upstream of the detection area, and are allowed to flow to the magnet, a majority of the particles are retained at a location where the laterally flowing solvent front first contacts the magnetic field.

In devices for measuring more than one analyte, one magnet may serve to provide a magnetic field for several discreet detection zones, such as one magnet underlying several discreet portions of the matrix, or each zone may have its own magnet. When more than one magnet is used, the field from one magnet should not attract particles that are intended to be attracted to the field of the other magnet(s).

Generally, the size of the device will provide for the ease of handling by an operator as well as provide sufficient signal from the labels such that the signals can be visually, photometrically, or spectrophotometrically detected. In on aspect, the porous carrier is a strip of about 6 mm by about 100 mm, and the device includes a rigid body of suitable size for holding the strip and the magnet in fixed relationship.

Suitable magnets are numerous. One example is a rare-earth neodymium-iron-boron rod magnet that is 0.25 inches×0.25 inches. This magnet has a strength of 40 MGOe (Mega Gauss Oersted). Other geometries including discs and cubes in other sizes are also appropriate. While different strength magnets are available, the distance of the magnet from the matrix affects the efficiency of the particle capture. For example, it has been found that when a 40MGOe magnet is placed 0.25 inches away from the matrix, only 0.8% of the particles seen when the magnet was touching the underside of the matrix.

The method and device includes the use of various reagents. These reagents may be added to the device independent of the sample, they may be added to mixtures containing the sample, or they may be stored on-board the device. The reagents include wash reagents for removing unbound reaction materials from the detection zone, detection reagents for detecting the presence of the analyte in the detection zone, and pre-wetting reagents that treat the porous matrix prior to the additional of the sample to reduce non-specific binding.

Wash reagents are well known to those of skill in the art of lateral flow devices. The reagent is capable of removing unbound reactants from the detection zone are appropriate. These reagents are generally a combination of low molecular weight carrier proteins, detergents and preservative. One such reagent is a component of the SNAP® FeLV/FIV Combo Assay (IDEXX Laboratories).

When the label on the conjugate reagent is an enzyme, the detection reagents may include a substrate which produces a detectable signal upon reaction with the enzyme in the detection zone. For example, the well-characterized enzyme horseradish peroxidase produces a colored product when reacted with the substrate, 4-chloro-1-napthol. One commercially-available substrate solution is TM Blue, which is available from TSI Incorporated (Worcester, Mass.). Also of interest are enzymes which involve the production of hydrogen peroxide and the use of the hydrogen peroxide to oxidize a dye precursor to a dye. Particular combinations include saccharide oxidases e.g., glucose and galactose oxidase, or heterocyclic oxidases, such as uricase and xanthine oxidase, coupled with an enzyme which employs the hydrogen peroxide to oxidize a dye precursor, e.g., peroxidase, microperoxidase, and cytochrome C oxidase. Other well known enzymatic reactions result in chemiluminescence (e.g., luminal and HRP) or fluorescence (e.g., methylumbelliferone and alkaline phosphatase) signals. In some instances, the wash reagent and the detection reagent are the same reagent, whereby the substrate reagent is capable of removing unbound reactants from the detection zone while also participating in the detection reaction.

The wash reagent and detection reagents may be stored on-board the device in breakable storage vessels as described in U.S. Pat. No. 5,726,010, which is incorporated herein by reference in its entirety. Reagents may be delivered to the porous matrix by a reagent delivery wick. The delivery wick may include a lance which serves to both pierce the storage vessels and deliver the reagent to the flow matrix. This linkage facilitates the release of the two stored liquid reagents with a single action. Sequential utilization of the two reagents, i.e., wash reagent followed by detector reagent may also be accomplished. Reagents may also be delivered through automated pipetting stations which dispense reagents onto the porous matrix at defined locations and at defined rates and volumes.

The device of the invention may also include an absorbent reservoir for absorbing the excess sample and reagents. Materials suitable for use as an absorbent reservoir are preferably highly absorbent, provide capacity in excess of the volume of the fluid sample plus the added liquid reagents, and are capable of absorbing liquids from the flow matrix by physical contact as the sole means of fluid transfer between the two materials. A variety of materials and structures are consistent with these requirements. Fibrous structures of natural and synthetic fibers such as cellulose and derivatized cellulose (e.g., cellulose acetate) are preferred for this use. The fibers of the material may be oriented along a particular axis (i.e., aligned), or they may be random. A preferred embodiment of the invention utilizes non-aligned cellulose acetate fibers of density range 0.1 to 0.3 grams per cubic centimeter and void volume of 60 to 95 percent. One such material is R-13948 Transorb Reservoir available from American Filtrona Corporation (Richmond, Va.).

Operation of the Device

The methods and devices of the invention facilitate sandwich or competition-type specific binding assays. In accordance with the invention, the analyte-specific binding partner is retained in the detection zone without the need of directly or indirectly attaching the partner to the matrix. In general, the particulate reagent can be added to the sample prior to adding the sample to the device. The conjugate reagent may be added to the sample, or may be added to the device after the particulate reagent has been reacted with the sample. Alternatively, the conjugate reagent may be present in a solubilizable form on the device such that the sample solution solubilizes the reagent. In another aspect, the particulate reagent may be added to the device prior to the addition of any reagents, including during the manufacturing of the device.

In the case of a sandwich assay, the analyte-specific binding reagent (e.g., an antibody) is immobilized in the detection zone as a result of its presence on the particulate reagent. Following binding of the sample analyte to its binding partner, the complex is detected with the label on the conjugate reagent. The analyte is sandwiched between the analyte-specific binding partners on the particulate reagent and on the conjugate reagent. The analyte-specific binding partners may be the same or different.

In the case of a competition assay, a number of assay formats are possible depending on the composition of the particulate reagent and the sequence of the assay. Competition formats include, for example, a one-step assay where the particulate reagent, containing an analyte-specific binding partner, is contacted simultaneously with a sample and a labeled analyte analog. A one-step assay can also be achieved where the analyte analog is on the particulate reagent and the analyte-specific binding partner is labeled with an appropriate reporter molecule. In a two-step competition assay, the sample is mixed with one assay component (e.g., labeled analyte analog) and then after a period of incubation it is mixed with the remaining assay component (i.e., particulate reagent having analyte-specific binding partner). As with a one-step assay, either the analyte analog or the analyte-specific binding partner can be located on the particulate reagent with the other being labeled. In an example of the two-step format, the analyte analog, located on the particulate reagent, is mixed with the sample for a period of time. A labeled analyte-specific binding partner is then added to the reaction mixture for a second incubation. Regardless of the assay format, the amount of label detected at the detection zone is inversely proportional to the amount of analyte in the sample.

Reagents and sample are contacted with the porous matrix at a sample application zone. The sample application zone is usually upstream of the detection zone so that the liquid reagents flow from the sample application zone to and through the detection zone as a result of the properties of the device for achieving lateral flow. Excess liquid may be captured in an absorbent reservoir. Wash and/or detection reagents may be added, or may be present on-board the device. No specific method of adding the sample and reagents to the device is required. The sample may be applied by dropping the liquid sample and reagents onto the device, or the device may be dipped into the reagents. The sample application zone may optionally include a separate matrix that contains dried reagents that are solubilized upon contact with the sample liquid. For example, the conjugate reagent may be present in a dried form and, when contacted with the sample liquid, become solubilized and participate in the detection reaction. In addition to being present in a separate matrix, the reagents may be present on the porous matrix at a location that allows the solubilization of the reagents by the sample liquid or other liquid such that the reagent can flow to the detection zone and participate in the detection reaction.

The sample application zone may partially or completely overlap the detection zone. However, it has been found that optimum performance of the device is achieved with the sample application zone is laterally spaced from the detection zone. The distance is desired to allow separation of bound and unbound material and thus reduce non-specific binding, for example between the matrix and conjugate reagent. In general, this distance is approximately between 4 and 10 mm. The distance is generally a compromise between longer distances that allow for greater separation and shorter distances that increase the speed of the assay due, in part, to the magnetic reagent's attraction to the magnet. The distance between the sample application zone and the detection zone may depend upon the material of the matrix. In one aspect of the invention using a porous carrier matrix having a lateral flow rate of 70 mm/4 min, the detection zone is about 10 mm from the sample application zone.

The matrix may be pre-wetted, i.e. before the addition of the sample, with a reagent that improves the hydrophobicity of the material. Pre-wetting reagents may be added to any part of the matrix as long as the reagents flow throughout the region including the application zone, the detection zone and the path in between the zones. Examples of pre-wetting reagents include buffers, detergents and low molecular weight carrier proteins, either alone or in a combination of two or three reagents in a premixed form.

Following the addition of the sample and reagents to the device, the label on the conjugate reagent is determined with the method appropriate for the label used. The device should provide an opening or a clear window in the area of the detection zone so that the signal from the labels can be detected visually or with any device capable of measuring or detecting light including, for example, photomultiplier tubes (PMTs), avalanche photodiodes (APDs) and charge-coupled devices (CCDs). When two labels are associated with the particulate reagent, for example when the label attached to the colloid is chemiluminescent label and the colloid has a fluorescent label bound via the conjugate reagent, the signals from the two labels are detected sequentially. The fluorescent label attached to the colloid is detected by exciting the label with the appropriate wavelength and detecting the emission of light. The chemiluminescent signal is read directly following the addition of the enzyme substrate. The same detection device can be used to measure both the fluorescent and the chemiluminescent signals or each signal can be read on independent detection systems. The detection system used may be the same or they may be of different types with the main requirement being that the sensitivity is sufficient to perform the assay.

Calibration

In one aspect, the invention is directed to method for calibrating an assay that does not rely on the standard practice of running calibration curves in tandem with or prior to the determination of analytes in unknown samples.

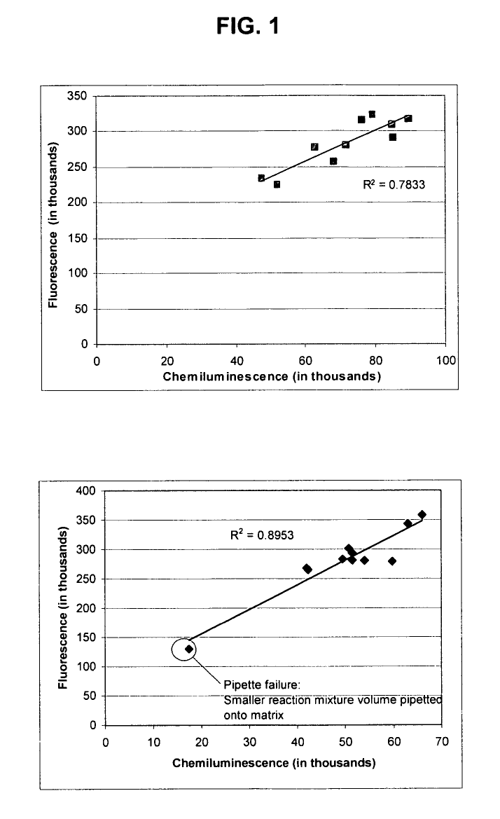

In this aspect, the invention allows for the quantification of the amount of particulate reagent independently of the signal associated with the presence of the analyte (analyte signal). For example, the label attached to the colloid provides a signal from the detection zone from which the amount of analyte-specific binding partners in the detection zone can be determined. Because the quantity of the particulate reagent in the detection zone is generally proportional to the assay signal, it is possible to account for small changes in signal from the analyte-specific binding partner that are due to small changes in the amount of particulate reagent within the detection area. For example, i.e. a 10% drop in a signal from an analyte-specific binding partner can be expected if there is a 10% drop in the signal from the label attached to the colloid. FIG. 1 shows the results of two experiments using various concentrations of a particulate reagent that includes, in these experiments, both the chemiluminescent and fluorescent labels (the particles were coated with HRP enzyme). The change fluorescent signal is proportional to the change in the chemiluminescent signal.

Having the label on the colloid of the particulate reagent, which provides a signal independent of a signal associate with the presence of the analyte in the sample (analyte signal), allows the assay to account for variation in analyte signal intensity arising from the variation arising from dilution and pipetting of the reagent, and from the potential variation of movement of the reagent within the matrix. Also, the calibration system also acts to alert the operator to catastrophic failures, such as a failure to transfer reagent or a failure to capture the particulate reagent in the detection zone.

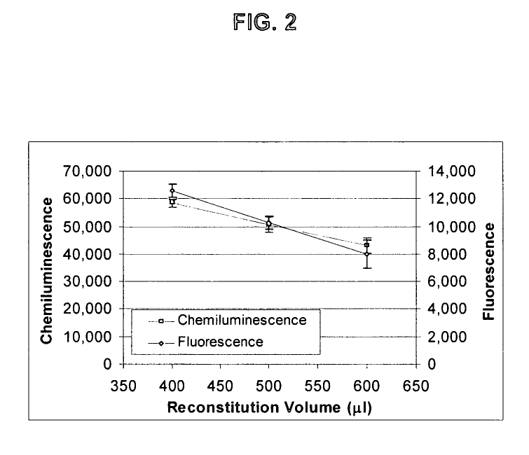

Equally important, the presence of the label on the colloid allows for the determination of the concentration of the particulate reagent after rehydration from a lyophilized state. For example, the invention allows the accurate determination of concentration of the rehydrated material without running a control assay or by using a label that can independently determine the concentration of the material. Using the invention, if the lyophilized particulate reagent is accidentally rehydrated with twice the volume of liquid required, the signal measured from the label attached to the colloid would be half that which would normally expected. This change in assay reagents could then be accounted for and the assay adjusted so that it still meets its operational parameters. For example, FIG. 2 shows that as the dilution of the lyophilized particulate reagent having both a fluorescent and chemiluminescent label (the chemiluminescent label was directly attached to the colloid). The volume of the particles prior to lyophilization was 500 μl. As the amount of reagent dilution increases, the amount of signal from the fluorescent label decreases. The decrease in the chemiluminescent label as the result of dilution is directly proportional to the decrease in the signal from fluorescent label.

In another aspect, a particulate reagent consisting of a magnetic particle labeled with an enzyme and a fluorescently labeled colloid may be used to calibrate the substrate used for the chemiluminescence signal. It is well known that such substrates degrade over time. As the substrate degrades, the slope of the dose response will also change. Using the above reagent, the dose response of the substrate can be determined, regardless of the degradation of the substrate. The enzyme signal should be titrated so that both the chemiluminescence and the fluorescence signals show a linear trend.

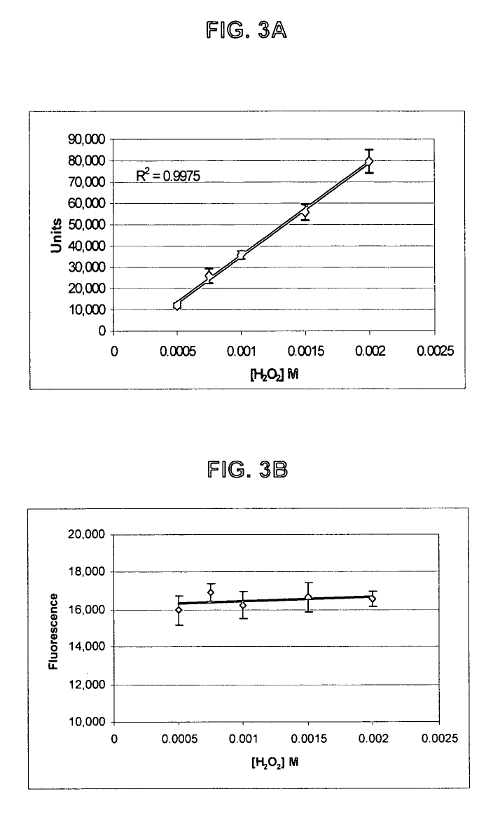

For example, FIG. 3A shows that as the active component of the substrate for HRP (hydrogen peroxide) is decreased, the chemiluminescent signal decreases. FIG. 3B shows that this decrease is not due to a difference in the amount of particulate reagent used since there is no change in fluorescent signal. Therefore, the change in the chemiluminescent signal can be determined to be solely due to a change in the substrate activity.

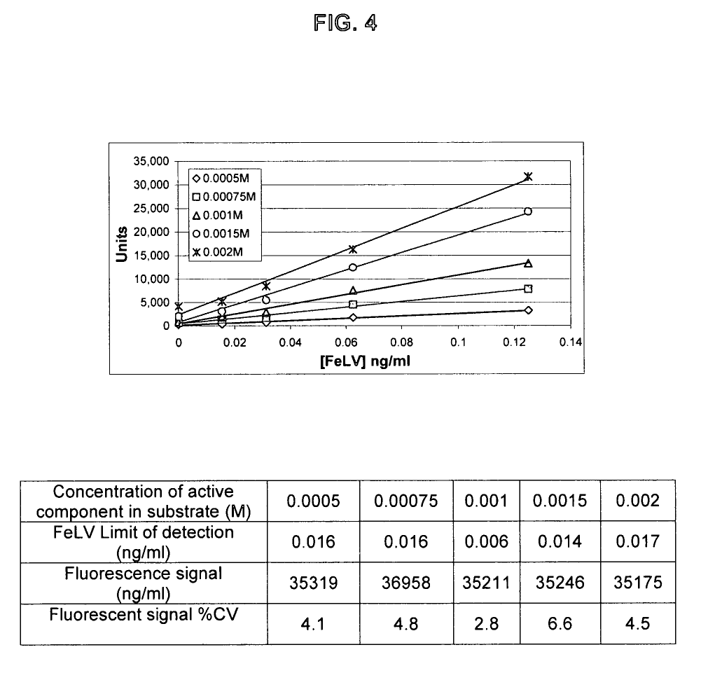

FIG. 4 shows how changes in substrate activity affect the dose response of the assay. The activity of the substrate can be measured using a fixed amount of particulate reagent, which is determined by the fluorescent signal. The measured activity can be compared to the activity of the substrate at the time of manufacture. Any significant change (degradation) can be accounted for in an adjustment in the standard curve used to determine the analyte.

Kits

Any or all of the above embodiments of the invention may be provided as a kit. In one particular example, such a kit would include a device of the invention complete with specific binding reagents, for example, non-immobilized conjugate reagent specific for analyte binding and a particulate reagent, as well as wash reagent and detector reagent. Positive and negative control reagents may also be included, if desired or appropriate. In addition, other additives may be included, such as stabilizers, buffers, and the like. The relative amounts of the various reagents may be varied widely, to provide for concentrations in solution of the reagents that substantially optimize the sensitivity of the assay. Particularly, the reagents may be provided as dry powders, usually lyophilized, which on dissolution will provide for a reagent solution having the appropriate concentrations for combining with the sample.

The following are provided for exemplification purposes only and are not intended to limit the scope of the invention described in broad terms above. All references cited in this disclosure are incorporated herein by reference.

### EXAMPLES

### Example 1

### Biotinylation of 200 nm Magnetic Particles

Carboxy functionalized 200 nm microparticles were biotinylated according to the following procedure.

Materials:

200 nM Carboxy Magnetic Particles 3.1% solids (Ademtech # 02123)

Biotin PEO-LC-Amine, fw 418.6 (Pierce # 21347)

MES, fw 195.2 (Sigma # M-8250)

Trisma Base, fw 121.1 (Sigma # T-1503)

Triton X-100, (Sigma # X-100)

EDC, (1-Ethyl-3-[3-dimethylaminopropyl]carbodiimide hydrochloride), fw 191.7

(Pierce #22980)

BSA, Bovine Serum Albumin (Ameresco #0332)

Prepare:

A. 50 mM MES Buffer pH 6.1:

Dissolve 9.76 g MES in 800 mL water. Adjust pH to 6.1. QS to 1.0 L
   B. 50 mM Tris Buffer pH 9.0:
Dissolve 6.06 g Tris in 800 mL water. Adjust pH to 9.0 QS to 1.0 L
   C. 50 mM Tris/1% Triton X-100 Buffer pH 9.0:
Dissolve 6.06 g Tris in 800 mL water. Add 10 mL Triton X-100. Adjust pH to 9.0 QS to 1.0 L
   D. 48 mM Biotin in MES Buffer:
Dissolve 32 mg Biotin PEO-LC-Amine in 1.6 mL MES Buffer.
   E. 50 mM EDC in MES Buffer:
Dissolve 34 mg in 3.55 mL MES Buffer.
   F. 5% Bovine Serum Albumin (BSA) in MES Buffer
Dissolve 5.0 g Bovine Serum Albumin in 80 ml, MES. Adjust pH to 6.1 QS to 100 mL.

Centrifuge 2.0 mL of 200 nm particles to pellet solids. Decant supernatant. Suspend particles in 15 mL 50 mM MES Buffer. Repeat solvent exchange (wash) twice more with 15 mL of 50 mM MES Buffer. Finally suspend Particles in 3.4 mL of 50 mM MES Buffer.

Add to Particles 1.3 mL of 48 mM Biotin-PEO-LC-Amine, then add 1.3 mL 50 mM EDC. Allow one hour with end over end rotation at room temperature. Add 0.6 mL of 5% BSA in MES Buffer. Allow one hour with end over end rotation at room temperature.

Centrifuge to pellet solids. Decant supernatant. Suspend particles in 15 mL 50 mM Tris/1% Triton Buffer. Repeat solvent exchange (wash) twice more with 15 mL of 50 mM Tris/1% Triton X-100 Buffer.

Centrifuge to pellet solids. Decant supernatant. Suspend particles in 15 mL 50 mM Tris Buffer. Repeat solvent exchange (wash) twice more with 15 mL of 50 mM Tris Buffer.

Finally suspend particles in 6.0 mL 50 mM Tris Buffer. Determine % Solids and Store at 4° C. until use.

These particles are now ready for fluorescent “staining” with Neutravidin coated fluorescent 15 nM colloidal gold particles.

### Example 2

### Preparation of a Dual Reactive (FeLV Antigen and Biotin Binding) Fluorescent 15 nM Colloidal Gold Reagent

Materials:

15 nm colloidal gold (British Biocell Inc.)

Anti FeLV monoclonal antibody (IDEXX Laboratories Inc.)I

Neutravidin (Pierce)

Europium Chloride, fw 258.3 (Aldrich, # 42,973-2)

Trioctylphosphine Oxide, fw 386.7 (Aldrich # 223301)

3,5, di-fluoro, phenyl, napthyl, propane dione, fw 310.3 (IDEXX Laboratories)

Polyethylene Glycol, fw˜15-20,000 (Sigma # P-2263)

Methanol (Sigma # 32,241-5)

Dioxane (Sigma #27,053-9)

Borax (Sigma B-3545)

Prepare:

A. 40 mM Borate Stock

15.25 g Borax dissolved in 800 mL water. QS to 1.0 L.
   B. 2 mM Borate Buffer pH 9.0
50 mL 40 mM Borate added to 800 mL water. pH to 9.0 QS to 1.0 L
   C. 10% Poly-Ethylene Glycol in water
Dissolve 10 g poly-ethylene Glycol in 80 mL water. QS to 100 mL.
   D. 0.3% Poly-Ethylene Glycol in 2 mM Borate
Add 50 mL 40 mM Borate and 30 mL 10% Poly-Ethylene Glycol to 800 mL water. Adjust pH to 7.1. QS to 1.0 L
   E. anti-FeLV Antibody
Desalt/Dialyze antibody into 2 mM Borate. Dilute antibody to 0.6 mg/mL (concentration optimized) with 2 mM Borate.
   F. Neutravidin
Dissolve Neutravidin in 2 mM Borate. Desalt/Dialyze Neutravidn into 2 mM Borate. Dilute Neutravidin to 2.3 mg/mL (concentration optimized) with 2 mM Borate.
   G. 20 mM Europium Chloride in 2 mm Borate
Dissolve 8.1 mg Europium. Chloride in 1.56 mL 2 mM Borate
   H. 40 mM Trioctylphosphine Oxide in Methanol
Dissolve 71.2 mg in 4.6 mL Methanol
   I. 20 mM 3,5, di-fluoro, phenyl, napthyl, propane dione in Dioxane
Dissolve 33.2 mg in 5.35 mL Dioxane
   J. 4 mM Europium Chelate in 60% Dioxane/20% Methanol/20% Water
To a glass tube add,
0.2 mL 40 mM Trioctylphosphine Oxide
0.6 mL 20 mM 3,5, di-fluoro, phenyl, napthyl, propane dione
0.2 mL 20 mM Europium Chloride

Procedure:

To 300 mL of 15 mM colloidal gold,

Add (drop-wise with rapid stirring):

0.75 mL of 4 mM Europium Chelate—Allow 5 minutes with mixing.

Add (drop-wise with rapid stirring):

3.0 mL of 0.6 mg/mL anti-FeLV Antibody—Allow 5 minutes with mixing.

Add (drop-wise with rapid stirring):

3.0 mL of 2.3 mg/mL Neutravidin—Allow 20 minutes with mixing.

Add (drop-wise with rapid stirring):

9.5 mL of 10% Poly-Ethylene Glycol—Allow 20 minutes with mixing. Centrifuge 15 nm gold (10.5 krpm, for 1 hour) to pellet gold particles. Decant supernatant and re-suspend in 300 mL of 0.3% Poly-Ethylene Glycol. Repeat solvent exchange (wash) twice more with 300 mL of 0.3% Poly-Ethylene Glycol. Finally suspend gold in 10 to 30 ml of 0.3% Poly-Ethylene Glycol. The now fluorescent 15 nm gold particles are ready for use.

### Example 3

### Specific Binding of Fluorescent Colloidal Gold Reagent to Magnetic Particles

Add 500 μl of 15 nm Neutravidin-IgG (anti-FELV) fluorescent colloidal gold prepared as in Example 2 to 50 μl of 200 nm to Biotin Magnetic Particles prepared as in Example 1. Vortex and incubate at room temperature for 1 hour. Following incubation, add 6.18 μl of 1 mg/ml Biotin solution (Pierce EZ-Link 5-(Biotinamido) pentylamine Part #21345 dilute with 50 mM TRIS/0.05% Tween pH 9). Place the solution on 1″ cubed magnet for 10 minutes to collect particles. Remove Supernatant.

Resuspend in 550 μl of 50 mM TRIS/0.05% Tween pH 9. Add 6.18 μl of 1 mg/ml Biotin solution. Capture particles on magnet as above. Remove supernatant. Resuspend as above and add biotin as above. Capture particles as above and remove supernatant. Resuspend and dilute to 0.04% solids (based on biotin magnetic particles) using TRIS buffer.

### Example 4

### Covalent Attachment of Fluorescent Colloidal Gold Reagents to Magnetic Particles

Materials:

200 nM Carboxy Magnetic Particles 3.1% solids (Ademtech # 02123)

15 nM Fluorescent Colloidal Gold Reagent (prepared as in Example 2 without the addition of neutravidin).

MES, fw 195.2 (Sigma # M-8250)

Trisma Base, fw 121.1 (Sigma # T-1503)

EDC (1-Ethyl-3-[3-dimethylaminopropyl]carbodiimide hydrochloride), fw 191.7 (Pierce # 22980)

Prepare:

A. 50 mM MES Buffer pH 6.1

Dissolve 9.76 g MES in 800 mL water. Adjust pH to 6.1. QS to 1.0 L

B. 50 mM Tris Buffer pH 9.0

Dissolve 6.06 g Tris in 800 mL water. Adjust pH to 9.0 QS to 1.0 L

C. 30 uM EDC in MES Buffer

Dissolve 7.3 mg EDC in 2.54 mL MES Buffer.

Dilute 1:500 into MES Buffer.

Procedure:

Centrifuge 0.09 mL of 200 nm particles to pellet solids. Decant supernatant. Suspend particles in 1.5 mL 50 mM MES Buffer. Repeat solvent exchange (wash) twice more with 1.5 mL of 50 mM MES Buffer. Suspend 200 nm particles in 0.3 mL of 50 mM MES Buffer.

Centrifuge 0.787 mL of 15 nm fluorescent particles to pellet solids. Decant supernatant. Suspend 15 nm particles in 0.3 mL of 50 mM MES Buffer. Combine 200 nm magnetic particles with 15 nm fluorescent particles. Add 0.3 mL of 30 uM EDC solution to particle mixture. Allow two hour with end over end rotation at room temperature.

Use magnetic separation to pellet solids. Decant supernatant. Suspend particle pellet in 1.0 mL 50 mM Tris Buffer. Repeat solvent exchange (wash) with magnetic separation until supernatant is clear. Finally suspend particles in 1.0 mL of 50 mM Tris Buffer. Determine % Solids and store at 4° C. until use.

### Example 5

### Analytical Device

A device was prepared using a 0.25 inch diameter, 40 MGOe rare-earth neodymium-iron-boron rod magnet fixed to a Porex matrix. which is a ultra high molecular weight polyethylene sheet material manufactured by Porex Technologies Corp. of Fairburn, Ga., USA. This material is made from fusing spherical particles of ultra-high molecular weight polyethylene (UHWM-PE) by sintering. This creates a porous structure with an average pore size of eight microns. The polyethylene surface is treated with an oxygen plasma and then coated with alternating layers of polyethylene imine (PEI) and poly acrylic acid (PAA) to create surfactant-free hydrophilic surface having wicking rate of 70 sec/4 cm. The matrix was cut into strips about 6.4 mm×100 mm. The magnet and matrix were held in place with a simple device holder.

### Example 6

### Detection of FELV (Flowing Reaction Mixture to the Magnet)

In the following example, 50 μl of a serum sample from a cat testing positive for leukemia virus using the IDEXX FeLV/FIV Combo SNAP® Test was mixed with 50 μl of Dual Immunoreactive/Fluorescent Particle prepared as in Example 3 or 4 and 12.5 μl of IgG HRP conjugate diluted at 20 μg/ml in a solution of preservatives, proteins, serum and buffer salts (e.g. Stabilzyme®, SurModics, Eden Prarie, Minn.).

The mixture was incubated at mixture for 10 min at 37° C. 15 μl of SNAP® wash reagent (IDEXX Laboratories) was spotted approximately 8.5 mm from the front tip of a Porex matrix in a device holder prepared as in Example 5. 5 μl of reaction mixture was spotted onto the Porex matrix approximately 10 mm in front of the 0.25″ diameter magnet. The magnet is approximately 20 mm from front tip of Porex matrix. 15 μl of SNAP® wash reagent is spotted a second time. To detect the signals, the matrix is placed in a reading device of either a luminometer or spectrophotometer. PS-atto substrate (Lumingen, Inc.) is flowed over the matrix to detect the chemiluminescent signal. Detection of the fluorescent label is achieved by shining a light source (e.g., a 365 nm LED) at the detection zone above the magnet, and light emitted from the fluorophore on the fluorescent colloidal gold is measured.

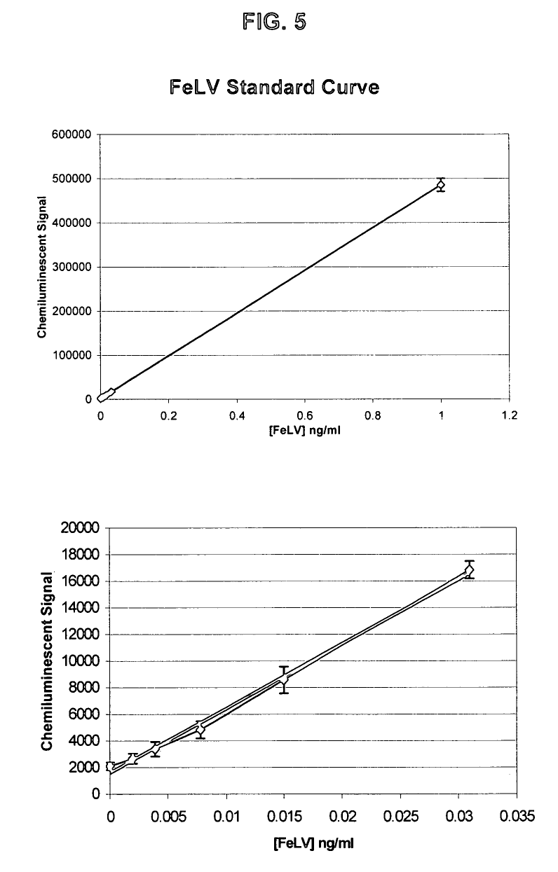

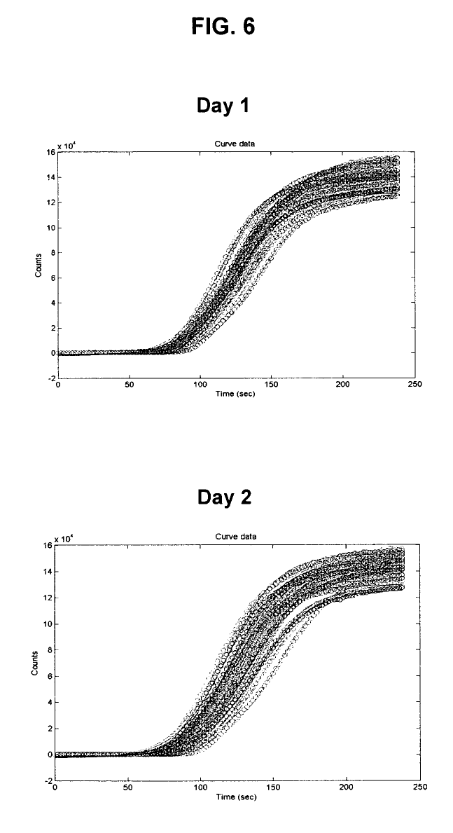

FIG. 5 shows the results of a number of assays for FeLV at various known concentrations of FeLV. The limit of detection was approximately 0.005 ng/ml. FIG. 6 shows the repeatability of the assay (30 assays conducted on two consecutive days) with an FELV concentration of 0.25 ng/ml. Assay error for all 60 assays was 6.6% (CV=6.6%).

### Example 7

### Detection of FeLV

A reaction mixture was prepared as in Example 6. The mixture was incubated for 10 minutes at 37° C. The Porex matrix in a device holder was prewetted for 20 seconds with the SNAP® wash reagent. 5 μl of reaction mixture was spotted onto the Porex matrix directly above the 0.25″ diameter magnet. The magnet is approximately 10 mm from tip of Porex matrix. SNAP® wash for flowed for 1 minute and the device was placed in a reader. Chemiluminescent and fluorescent signals were detected as in Example 6.

### Example 8

### Detection of FeLV

50 μl of the feline sample was mixed with 50 μl of 200 nm Biotin Magnetic Particle prepared as in Example 1, and 12.5 μl of IgG G3 HRP conjugate, and 13 μl of 15 nm Neutravidin-IgG fluorescent colloidal gold prepared as in Example 3. The mixture was incubated for 10 min at 37° C. A Porex matrix in a device holder was prewetted by spotting 15 μl of SNAP® wash reagent approximately 8.5 mm from front tip of the matrix. 5 μl of reaction mixture was spotted onto Porex matrix approximately 10 mm in front of 0.25″ diameter magnet. The magnet is approximately 20 mm from front tip of the matrix. 15 μl of SNAP® wash was spotted a second time. Chemiluminescent and fluorescent signals were detected as in Example 6.

### Example 9

### Detection of FeLV (Direct Deposition of Reaction Mixture on Magnet)

A reaction mixture and device were prepared as in Example 8. The Porex matrix was prewetted for 20 second with SNAP® was reagent. 5 μl of reaction mixture was spotted onto the Porex matrix directly above the 0.25″ diameter magnet. The magnet was approximately 10 mm from tip of the matrix. SNAP® wash reagent was flowed over the matrix for 1 min. Chemiluminescent and fluorescent signals were detected as in Example 6.

### Example 10

### Detection of T4

10 μl of sample having various concentrations of T4 was mixed with 20 μl of 0.05 μg/ml anti-T4 antibody conjugated HRP, the antibody conjugate being made in-house using antibody obtained from Fitzgerald International Industries (#M94207), and incubated at 37° C. for 5 minutes. 10 μl of magnetic particles coated with T3 and incubated for a further 5 minutes. A Porex matrix in a device holder was prewetted for 20 seconds with SNAP® wash reagent. 5 μl of reaction mixture was spotted onto the Porex matrix approximately 10 mm in front of a 0.25″ diameter magnet. The magnet is approximately 20 mm from front tip of Porex matrix. 15 μl of SNAP® wash reagent was spotted a second time. Chemiluminescence was detected as in Example 6.

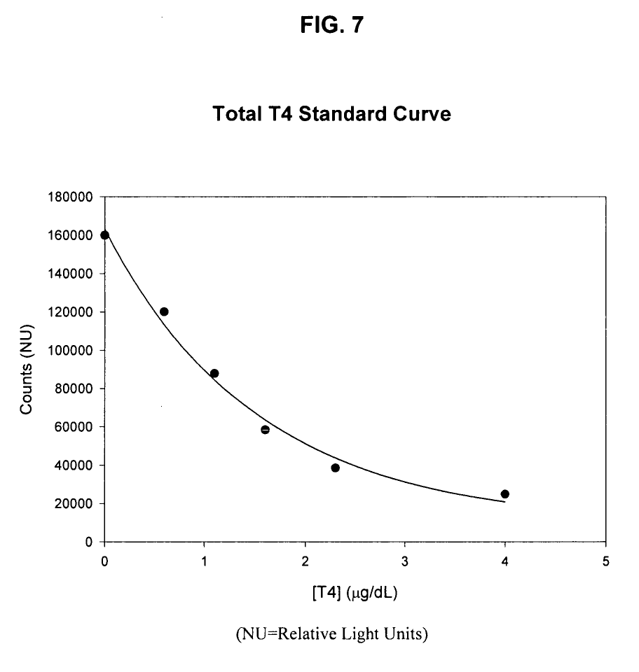

FIG. 7 shows the results of the T4 assay with various sample concentrations of T4.

Although various specific embodiments of the present invention have been described herein, it is to be understood that the invention is not limited to those precise embodiments and that various changes or modifications can be affected therein by one skilled in the art without departing from the scope and spirit of the invention.

1. A reagent comprising a complex comprising a magnetic nanoparticle bound to a metal colloid.

2. The reagent of claim 1 wherein the magnetic nanoparticle comprises an iron oxide and a polymer.

3. The reagent of claim 1 wherein the magnetic nanoparticle has a diameter of about 50-1000 nanometers.

4. The reagent of claim 3 wherein the magnetic nanoparticle has a diameter of about 100-500 nanometers.

5. The reagent of claim 1 wherein the colloid is covalently bound to the nanoparticle.

6. The reagent of claim 1 wherein the magnetic nanoparticle comprises a first specific binding partner, and the colloid comprises a second specific binding partner that is specific for the first specific binding partner.

7. The reagent of claim 6 wherein either (i) the first specific binding partner is biotin and the second specific binding partner is selected from the group consisting of avidin, streptavidin and neutravidin, or (ii) the first specific binding partner is selected from the group consisting of avidin, streptavidin and neutravidin, and the second specific binding partner is biotin.

8. The reagent of claim 2 wherein the polymer comprise an olefinic polymer or copolymer.

9. The reagent of claim 2 wherein the polymer comprises a polysaccharide.

10. The reagent of claim 1 where the magnetic nanoparticle comprises a functional group selected from the group consisting of hydroxyl, carboxyl, aldehyde, or amino.

11. The reagent of claim 1 wherein the metal is gold, silver or a rare earth metal.

12. The reagent of claim 1, further comprising a first label attached to the colloid.

13. The reagent of claim 12 wherein the first label comprises a fluorescent metal chelate.

14. The reagent of claim 1 wherein the nanoparticle or the colloid comprises an analyte-specific binding partner.

15. The reagent of claim 14 further comprising a first label attached to the colloid.

16. The complex reagent of claim 15 wherein the first label is a fluorescent metal chelate.

17-20. (canceled)

21. A particulate reagent for detecting an analyte in a sample comprising the reagent of claim 14, wherein the colloid further comprises a first label attached to the colloid.

22. The reagent of claim 21 wherein the metal is gold, silver or a rare earth metal.

23. The reagent of claim 21 wherein the label comprises a fluorescent metal chelate.

24. The reagent of claim 21 wherein the magnetic nanoparticle comprises an iron oxide and a polymer.

25. The reagent of claim 21 wherein the magnetic nanoparticle has a diameter of about 50-1000 nanometers.

26. The reagent of claim 21 wherein the magnetic nanoparticle has a diameter of about 100-500 nanometers.

27. The reagent of claim 21 wherein the colloid is covalently bound to the nanoparticle.

28. The reagent of claim 21 wherein the particle comprises a first specific binding partner, and the colloid comprises a second specific binding partner that is specific for the first specific binding partner.

29. The reagent of claim 24 wherein the polymer comprise an olefinic polymer or copolymer.

30. The reagent of claim 24 wherein the polymer comprises a polysaccharide.

31. The reagent of claim 24 where the magnetic nanoparticle comprises a functional group selected from the group consisting of hydroxyl, carboxyl, aldehyde, or amino.

32. A lyophilized reagent comprising the reagent of claim 24.

33-107. (canceled)
